(19) United States
(12) Patent Application Publication
(10) Pub. No.: US 2019/0023672 A1
(43) Pub. Date: Jan. 24, 2019

(54) METHODS FOR DIRECT EPOXIDATION OF PROPYLENE WITH OXYGEN

(71) Applicant: SABIC Global Technologies B.V., Bergen op Zoom (NL)

(72) Inventors: Tjeerd Alexander Nijhuis, Elsloo (NL); Ihab N. Odeh, Sugar Land, TX (US)

(21) Appl. No.: 16/071,954

(22) PCT Filed: Dec. 21, 2016
(86) PCT No.: PCT/IB2016/057908

§ 371 Date: Jul. 23, 2018

Related U.S. Application Data
(60) Provisional application No. 62/289,626, filed on Feb. 1, 2016.

(51) Int. Cl. C07D 301/10; C07D 303/04; B01J 23/52; B01J 19/24

(52) U.S. Cl. CPC C07D 301/10; C07D 303/04; B01J 23/52; B01J 2523/47; B01J 19/2415; B01J 2219/00042; B01J 2523/19; B01J 19/249

(57) ABSTRACT

Methods to produce propylene oxide are described. One method can include providing a propene feedstream, an oxygen feed stream and, optionally, a hydrogen feed stream to a reaction zone, and maintaining, in a reaction zone during the reaction, at least 50 vol. % propene and 1 to 15 vol. % O₂ by gradually introducing a feed stream that includes the O₂ over the length of the catalytic bed or the length of the reaction zone and/or a feed stream that includes the H₂ over the length of the catalytic bed or the length of the reaction zone.

### CROSS REFERENCE TO RELATED APPLICATIONS

This application claims the benefit of priority of U.S. Provisional Patent Application No. 62/289,626, filed Feb. 1, 2016, which is hereby incorporated by reference in its entirety.

### BACKGROUND OF THE INVENTION

### A. Field of the Invention

The invention generally concerns methods for the direct of epoxidation of propylene with oxygen and optionally hydrogen. In a particular aspect, the methods allow for the ability to safely provide oxygen, propylene, and optionally hydrogen to a reactor system and produce propylene oxide. The reaction conditions, which include (1) at least 50 vol. % propene, 1 to 15 vol. % O₂, and optionally 1 to 15 vol. % H₂ and (2) a temperature of 150° C. to 300° C. and a pressure of 3 bar to 20 bar, remain outside of the explosive regime due to the reactor configuration.

### B. Description of Related Art

Propylene oxide (PO) is an important chemical intermediate for the production of numerous commercial materials. For example, PO can be used to make propylene glycols and polyether polyols (e.g., polyglycol ethers, propylene glycol ethers, etc.). These chemical compounds can be used many commercial applications such as rigid foam insulations, flexible foam applications, flame retardants, starches, synthetic lubricants, oil field drilling chemicals, textile surfactants, food product additives, cosmetic applications, and the like.

One conventional method to make PO includes the reaction of propylene with chlorohydrin as shown in reaction Scheme I.

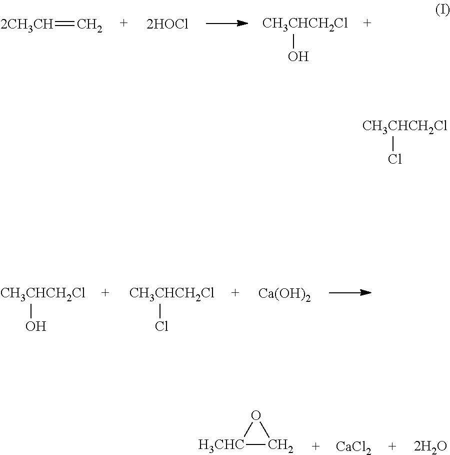

This method suffers from environmental liabilities and has high capital costs. These plants are often integrated with chloro-alkali plants to make the chlorine and caustic soda, which consume a large amount of power. Further, extensive effluent treatment may be needed to handle the large dilute calcium chloride brine waste stream.

Another conventional method to make PO includes a hydroperoxide method using, for example, cumene hydroperoxide as shown in reaction Scheme (II) or analogues thereof.

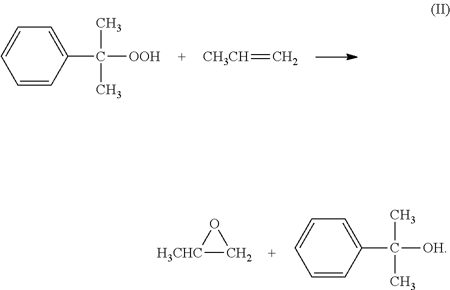

This reaction suffers in that the co-product a benzyl alcohol (alpha, alpha-dimethylbenzyl alcohol) must be isolated, and through a series of chemical steps converted to cumene, which is then oxidized by oxygen to cumene hydroperoxide.

Yet another conventional method to make PO includes the oxidation of propylene with hydrogen peroxide as shown in reaction Scheme (III). Commercially, propylene oxide can be made using anthraquinone to generate hydrogen peroxide. An alkylanthraquinone precursor dissolved in a mixture of organic solvents followed by liquid-liquid extraction to recover H₂O₂. The AO process is a multistep method that requires significant energy input and generates waste, which has a negative effect on its sustainability and production costs. The hydrogen peroxide is reacted with the propylene to make PO and water (HPPO process). This method suffers in that it requires significant energy input and generates waste, which has a negative effect on its sustainability and production costs

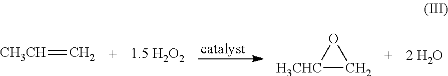

To overcome the limitations of the aforementioned conventional processes, direct epoxidation of propylene as shown in reaction Scheme (IV) using a wide variety of heterogeneous and homogeneous catalysts has been investigated. These methods suffer in that undesirable by-products (side products not shown in Scheme IV) or high amounts of water are produced, both of which result in low yields of propylene oxide.

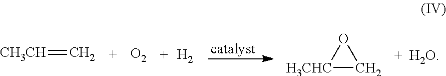

Still further, the above direct epoxidation of propylene reaction is typically carried out with dilute gas mixtures in which each of the reactants (propylene, oxygen, and hydrogen) is present at concentrations of 10 vol. % or lower and operated at relatively low pressures (typically around 1 bar) and/or temperatures (typically less than 150° C.). These conditions are used to avoid running the reaction within an explosive regime of the reactants. While this can be safely performed, a downside is decreased efficiency of the reaction.

There has been at least one attempt to run the direct epoxidation of propene reaction in the explosive regime (See, Nijhuis et al., “The Direct Epoxidation of Propene in the Explosive Regime in a Microreactor—A Study into the Reaction Kinetics”, Ind. Eng. Chem. Res., 2010, 49, 10479-85). However, the reactor used in Nijhuis et al. was a microreactor, which prevents scalability to commercial production of propylene oxide.

### SUMMARY OF THE INVENTION

A solution to the problems of direct epoxidation of propylene has been discovered. The solution is premised on the ability to safely provide oxygen, propylene, and optionally hydrogen to a reactor system and produce propylene oxide. The reaction conditions, which include (1) at least 50 vol. % propene, 1 to 15 vol. % O₂, and optionally 1 to 15 vol. % H₂ and (2) a temperature of 150° C. to 300° C. and a pressure of 3 bar to 20 bar, remain outside of the explosive regime due to the reactor configuration. In particular, the reaction can be safely performed under such reaction conditions by introducing propene through a first reactant feed stream and manipulating the introduction of oxygen gas (O₂) and optionally hydrogen gas (H₂) through separate feed streams over the length of the catalytic bed or the length of the reaction zone. By way of example, the O₂ feed stream and optionally the H₂ feed stream can be gradually (either incrementally, intermittently or both) introduced over the length of the catalytic bed or reaction zone such that an explosive concentration of propene, O₂, and H₂ (if present) is not exceeded at any point throughout the length of the reaction zone. Without wishing to be bound by theory, it is believed that these reaction parameters and conditions allow for the use of an overall explosive regime within a given reactor, but reduces the possibility of exceeding an explosive concentration of the reactants at any point within the reaction zone. This has the advantage of being able to safely operate the direct epoxidation of propene reaction under conditions that can maximize the efficiency of the reaction (e.g., reduced water production, increased propylene oxide yield, decreased production of by-products, and increased catalyst stability). Notably, any type of epoxidation catalyst can be used with the process of the present invention, as the improvements offered by the present invention focus on the reaction conditions rather than a specific catalyst.

In one aspect of the present invention, a method for direct epoxidation of propylene is described. The method can include reacting, in a reaction zone of a reactor (e.g., a continuous flow reactor selected from a fixed-bed reactor, a fluidized reactor, a plate reactor, a membrane reactor or a moving bed reactor), propene, oxygen gas (O₂), and hydrogen gas (H₂) in the presence of a catalytic bed that includes a propene epoxidation catalyst to produce a product stream that includes propylene oxide and, in some instances, water. At least 50 vol. % propene, 1 to 15 vol. % O₂, and 1 to 15 vol. % H₂ can be maintained in the reaction zone during the reaction by (i) introducing the propene through a first reactant feed stream and (ii) gradually introducing the O₂ or the H₂, or both, over the length of the catalytic bed or the length of the reaction zone through a separate reactant feed stream(s). The reaction can be performed at a temperature of 150° C. to 300° C., preferably 200° C. to 275° C., and a pressure of 3 bar to 20 bar, preferably 5 to 15 bar. In a particular aspect, 82 vol. % to 95 vol. % of propene, preferably 88 vol. % to 92 vol. % of propene, 3 vol. % to 8 vol. % O₂, preferably 4 vol. % to 6 vol. % O₂, and 2 vol. % to 10 vol. % H₂, preferably, 4 vol. % to 6 vol. % H₂, can be maintained in the reaction zone during the reaction. While the overall vol. % of propene, O₂, and/or H₂ in the reaction zone can be within the explosive regime/range, an explosive concentration of these reactants is not exceeded at any point throughout the length of the reaction zone due to the gradual introduction of O₂ and/or H₂ into the reaction zone. Gradual introduction can be performed through incremental introduction or intermittent introduction, or both, over the length of the catalytic bed or the length of the reaction zone. In some instances, H₂ is introduced gradually over the length of the catalytic bed or the length of the reaction zone in a second reactant feed stream through a membrane that is positioned proximate to the catalytic bed and/or O₂ is introduced over the length of the catalytic bed or the length of the reaction zone in a third feed stream through a second membrane that is positioned proximate to the catalytic bed. In one instances, O₂ or H₂ is introduced with the propene in the first reactant feed stream. The first reactant feed stream can be introduced through an inlet positioned upstream from the catalytic bed. In some instances, the separate reactant feed stream that gradually introduces O₂ or H₂, or both, over the length of the catalytic bed or the length of the reaction zone is introduced through a second inlet that is positioned downstream from the first inlet. The H₂ can be gradually introduced over the length of the catalytic bed or the length of the reaction zone through a second inlet that can be positioned downstream from the first inlet and the H₂ is introduced over the length of the catalytic bed or the length of the reaction zone through a third inlet that can be positioned downstream from the first inlet. In a particular instance the gradual introduction of O₂ or H₂, or both can be accomplished with a reactor having alternating plates. This can be accomplished, for instance, by using a reactor that includes alternating plates of an oxygen feed plate, an epoxidation catalyst plate, a hydrogen feed plate, a second epoxidation catalyst plate, a second oxygen feed plate, a third epoxidation catalyst plate, a second hydrogen feed plate, etc. The first reactant feed stream can include an inert gas (e.g., helium, nitrogen, argon, water vapor, carbon dioxide or any combination thereof) that is not involved/not consumed with the epoxidation of propene reaction. The reaction conditions can include a weight hourly space velocity (WHSV) of between 1 and 200 h⁻¹. The propylene epoxidation catalyst can be in particulate or powdered form. In other instances, the propylene epoxidation catalyst can be formed to have a selected shape (e.g., extrudated catalysts, spherically catalysts, pellets, or structured catalysts such as monoliths, foams, etc.). The catalyst can include titanium, gold, palladium, platinum, silver or any combination or alloy thereof. Still further, the propylene epoxidation catalyst can be supported by a support material (e.g., metal oxide supports such as silica or titania, zeolite supports such as TS-1, Ti-Beta, etc.). In general, any known propylene epoxidation catalyst can be used in the context of the methods of the present invention.

In another embodiment of the present invention there is disclosed a method for direct epoxidation of propene without using hydrogen gas (H₂) as a reactant. The method can include reacting, in a reaction zone of a reactor, propene and oxygen gas (O₂) in the presence of a catalytic bed that includes a propene epoxidation catalyst to produce a product stream comprising propylene oxide, where (1) at least 50 vol. % propene and 1 to 15 vol. % O₂ is maintained in the reaction zone during the reaction by (i) introducing the propene through a first reactant feed stream and (ii) gradually introducing the O₂ over the length of the catalytic bed or the length of the reaction zone through a separate reactant feed stream; (2) a temperature of 150° C. to 300° C. and a pressure of 3 bar to 20 bar is maintained in the reaction zone during the reaction; and (3) the direct epoxidation of propene reaction is performed in the absence of H₂ gas. Other than the absence of hydrogen gas in the reaction, the reaction can occur under the same parameters and processing conditions as those discussed above and throughout the present invention. Still further, this reaction can be performed in the complete absence of H₂ or with non-reactive amounts of H₂, such that H₂ is not present in an amount to effect the overall production of propylene oxide.

In the context of the present invention 43 embodiments are described. The first embodiment describes a method for direct epoxidation of propene. The method can include reacting, in a reaction zone of a reactor, propene, oxygen gas (O₂), and hydrogen gas (H₂) in the presence of a catalytic bed that includes a propene epoxidation catalyst to produce a product stream comprising propylene oxide, where at least 50 vol. % propene, 1 to 15 vol. % O₂, and 1 to 15 vol. % H₂ is maintained in the reaction zone during the reaction by (i) introducing the propene through a first reactant feed stream and (ii) gradually introducing the O₂ or the H₂, or both, over the length of the catalytic bed or the length of the reaction zone through a separate reactant feed stream(s), and a temperature of 150° C. to 300° C. and a pressure of 3 bar to 20 bar is maintained in the reaction zone during the reaction. Embodiment 2 is the method of embodiment 1, wherein 82 vol. % to 95 vol. % of propene, 3 vol. % to 8 vol. % O₂, and 2 vol. % to 10 vol. % H₂ is maintained in the reaction zone during the reaction. Embodiment 3 is the method of embodiment 2, wherein 88 vol. % to 92 vol. % of propene, 4 vol. % to 6 vol. % O₂, and 4 vol. % to 6 vol. % H₂ is maintained in the reaction zone during the reaction. Embodiment 4 is the method of any one of embodiments 1 to 3, wherein the vol. % of propene and O₂ or H₂, or both, in the reaction zone has an explosive regime, and wherein the gradual introduction of O₂ or H₂, or both, in the reaction zone is such that an explosive concentration of propene and O₂ or H₂, or both, is not exceeded at any point throughout the length of the reaction zone. Embodiment 5 is the method of any one of embodiments 1 to 4, wherein a temperature of 200° C. to 275° C. and a pressure of 5 bar to 15 bar is maintained in the reaction zone during the reaction. Embodiment 6 is the method of any one of embodiments 1 to 5, wherein O₂ or H₂, or both, are introduced incrementally over the length of the catalytic bed or the length of the reaction zone. Embodiment 7 is the method of any one of embodiments 1 to 5, wherein O₂ or H₂, or both, are introduced intermittently over the length of the catalytic bed or the length of the reaction zone. Embodiment 8 is the method of any one of embodiments 1 to 7, wherein H₂ is introduced gradually over the length of the catalytic bed or the length of the reaction zone in a second reactant feed stream through a membrane that is positioned proximate to the catalytic bed. Embodiment 9 is the method of embodiment 8, wherein O₂ is introduced over the length of the catalytic bed or the length of the reaction zone in a third feed stream through a second membrane that is positioned proximate to the catalytic bed. Embodiment 10 is the method of embodiment 8, wherein O₂ is introduced with the propene in the first reactant feed stream. Embodiment 11 is the method of any one of embodiments 1 to 7, wherein O₂ is introduced gradually over the length of the catalytic bed or the length of the reaction zone in a second reactant feed stream through a membrane that is positioned proximate to the catalytic bed. Embodiment 12 is the method of embodiment 11, wherein H₂ is introduced with the propene in the first feed stream. Embodiment 13 is the method of any one of embodiments 1 to 12, wherein the first reactant feed stream further comprises an inert gas. Embodiment 14 is the method of embodiment 13, wherein the inert gas is helium, nitrogen, argon, water vapor, carbon dioxide or any combination thereof. Embodiment 15 is the method of any one of embodiments 1 to 14, wherein the reaction conditions include a weight hourly space velocity (WHSV) of between 1 and 200 h⁻¹. Embodiment 16 is the method of any one of embodiments 1 to 15, wherein the product stream further comprises water. Embodiment 17 is the method of any one of embodiments 1 to 16, wherein the propene epoxidation catalyst comprises titanium, gold, palladium, platinum or any combination or alloy thereof. Embodiment 18 is the method of embodiment 17, wherein the propene epoxidation catalyst is supported with a metal oxide or a zeolite. Embodiment 19 is the method of any one of embodiments 1 to 18, wherein the propene epoxidation catalyst is in particulate form or formed to have a selected shape. Embodiment 20 is the method of any one of embodiments 1 to 19, wherein the first reactant feed stream is introduced through an inlet positioned upstream from the catalytic bed. Embodiment 21 is the method of any one of embodiments 1 to 20, wherein the separate reactant feed stream that gradually introduces O₂ or H₂, or both, over the length of the catalytic bed or the length of the reaction zone is introduced through a second inlet that is positioned downstream from the first inlet. Embodiment 22 is the method of embodiment 21, wherein the hydrogen gas is gradually introduced over the length of the catalytic bed or the length of the reaction zone through a second inlet that is positioned downstream from the first inlet. Embodiment 23 is the method of embodiment 22, wherein the oxygen gas is introduced over the length of the catalytic bed or the length of the reaction zone through a third inlet that is positioned downstream from the first inlet. Embodiment 24 is the method of any one of embodiments 1 to 23, wherein the reactor comprises alternating plates or tubes of an oxygen feed plate or tube, an epoxidation catalyst plate or tube, and a hydrogen feed plate or tube. Embodiment 25 is the method of any one of embodiments 1 to 24, wherein the reaction zone is a continuous flow reactor selected from a fixed-bed reactor, a fluidized reactor, a plate or tube reactor, a membrane reactor or a moving bed reactor.

Embodiment 26 is a method for direct epoxidation of propene without using hydrogen gas (H₂) as a reactant, the method comprising reacting, in a reaction zone of a reactor, propene and oxygen gas (O₂) in the presence of a catalytic bed that includes a propene epoxidation catalyst to produce a product stream comprising propylene oxide, where at least 50 vol. % propene and 1 to 15 vol. % O₂ is maintained in the reaction zone during the reaction by (i) introducing the propene through a first reactant feed stream and (ii) gradually introducing the O₂ over the length of the catalytic bed or the length of the reaction zone through a separate reactant feed stream; a temperature of 150° C. to 300° C. and a pressure of 3 bar to 20 bar is maintained in the reaction zone during the reaction; and the direct epoxidation of propene reaction is performed in the absence of H₂ gas. Embodiment 27 is the method of embodiment 26, wherein 82 vol. % to 95 vol. % of propene and 3 vol. % to 8 vol. % O₂ is maintained in the reaction zone during the reaction. Embodiment 28 is the method of embodiment 26, wherein 88 vol. % to 92 vol. % of propene and 4 vol. % to 6 vol. % O₂ is maintained in the reaction zone during the reaction. Embodiment 29 is the method of any one of embodiments 26 to 28, wherein the vol. % of propene and O₂ in the reaction zone has an explosive regime, and wherein the gradual introduction of O₂ in the reaction zone is such that an explosive concentration of propene and O₂ is not exceeded at any point throughout the length of the reaction zone. Embodiment 30 is the method of any one of embodiments 26 to 29, wherein a temperature of 200° C. to 275° C. and a pressure of 5 bar to 15 bar is maintained in the reaction zone during the reaction. Embodiment 31 is the method of any one of embodiments 26 to 30, wherein O₂ is introduced incrementally or intermittently over the length of the catalytic bed or the length of the reaction zone. Embodiment 32 is the method of any one of embodiments 26 to 30, wherein O₂ is introduced gradually over the length of the catalytic bed or the length of the reaction zone in a second reactant feed stream through a membrane that is positioned proximate to the catalytic bed. Embodiment 33 is the method of any one of embodiments 26 to 32, wherein the first reactant feed stream further comprises an inert gas. Embodiment 34 is the method of embodiment 33, wherein the inert gas is helium, nitrogen, argon, carbon dioxide or any combination thereof. Embodiment 35 is the method of any one of embodiments 26 to 34, wherein the reaction conditions include a weight hourly space velocity (WHSV) of between 1 and 200 h⁻¹. Embodiment 36 is the method of any one of embodiments 26 to 35, wherein the product stream further comprises water. Embodiment 37 is the method of any one of embodiments 26 to 36, wherein the propene epoxidation catalyst comprises titanium, gold, palladium, platinum or any combination or alloy thereof. Embodiment 38 is the method of embodiment 37, wherein the propene epoxidation catalyst is supported with a metal oxide or a zeolite. Embodiment 39 is the method of any one of embodiments 26 to 38, wherein the propene epoxidation catalyst is in particulate form or formed to have a selected shape. Embodiment 40 is the method of any one of embodiments 26 to 39, wherein the first reactant feed stream is introduced through an inlet positioned upstream from the catalytic bed. Embodiment 41 is the method of any one of embodiments 26 to 40, wherein the separate reactant feed stream that gradually introduces O₂ over the length of the catalytic bed or the length of the reaction zone is introduced through a second inlet that is positioned downstream from the first inlet. Embodiment 42 is the method of any one of embodiments 26 to 41, wherein the reactor comprises alternating plates or tubes of an oxygen gas feed plate or tube and an epoxidation catalyst plate or tube. Embodiment 43 is the method of any one of embodiments 26 to 42, wherein the reaction zone is a continuous flow reactor selected from a fixed-bed reactor, a fluidized reactor, a plate or tube reactor, a membrane reactor or a moving bed reactor.

The following includes definitions of various terms and phrases used throughout this specification.

The terms “propene” and “propylene” refer to a compound having the structure CH₃CH═CH₂.

The term “inert” is defined as chemically inactive or substantially inactive under the reaction conditions. Non-limiting examples of inert chemical compounds in the context of this invention include helium, nitrogen, argon, and carbon dioxide.

The terms “about” or “approximately” are defined as being close to as understood by one of ordinary skill in the art. In one non-limiting embodiment, the terms are defined to be within 10%, preferably within 5%, more preferably within 1%, and most preferably within 0.5%.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The terms “wt. %”, “vol. %”, or “mol. %” refers to a weight, volume, or molar percentage of a component, respectively, based on the total weight, the total volume of material, or total moles, that includes the component unless otherwise stated. In a non-limiting example, 10 grams of component in 100 grams of the material is 10 wt. % of component.

The use of the words “a” or “an” when used in conjunction with any of the terms “comprising,” “including,” “containing,” or “having” in the claims, or the specification, may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The methods of the present invention can “comprise,” “consist essentially of,” or “consist of” particular ingredients, components, compositions, etc. disclosed throughout the specification. With respect to the transitional phrase “consisting essentially of,” in one non-limiting aspect, a basic and novel characteristic of the methods of the present invention is the ability to safely produce of propylene oxide under reaction conditions that include (1) at least 50 vol. % propene, 1 to 15 vol. % O₂, and optionally 1 to 15 vol. % H₂ and (2) a temperature of 150° C. to 300° C. and a pressure of 3 bar to 20 bar.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

Advantages of the present invention may become apparent to those skilled in the art with the benefit of the following detailed description and upon reference to the accompanying drawings.

FIG. 1A is a schematic of a continuous flow reactor system to produce propylene oxide using the method of the present invention that includes a propene feed stream, an oxygen feed stream and a hydrogen feed stream.

FIG. 1B is a schematic of a continuous flow reactor system to produce propylene oxide using the method of the present invention that includes a propene feed stream, an oxygen feed stream and a hydrogen feed stream and staggered hydrogen and oxygen outlets.

FIG. 1C is a schematic of a continuous flow reactor system to produce propylene oxide using the method of the present invention with a propene feed stream and an oxygen feed stream.

FIG. 2A is a schematic of a membrane reactor system for producing propylene oxide using the methods of the present invention.

FIG. 2B is a cross-sectional view of the system shown in FIG. 2A.

FIG. 3A is a schematic of a fluidized reactor system to produce propylene oxide using the method of the present invention that includes a propene feed stream, an oxygen feed stream and a hydrogen feed stream.

FIG. 3B is a schematic of a fluidized flow reactor system to produce propylene oxide using the method of the present invention that includes a propene feed stream, an oxygen feed stream and a hydrogen feed stream and staggered hydrogen and oxygen injectors.

FIG. 3C is a schematic of a fluidized flow reactor system to produce propylene oxide using the method of the present invention with a propene feed stream and an oxygen feed stream.

FIG. 4A is a schematic of a fluidized reactor system to produce propylene oxide using the method of the present invention that includes a propene feed stream, an oxygen feed stream and a hydrogen feed stream, where the oxygen feed stream and the hydrogen feed stream flow perpendicular to the propene feed stream.

FIG. 4B is a schematic of a fluidized flow reactor system to produce propylene oxide using the method of the present invention that includes a propene feed stream, an oxygen feed stream and a hydrogen feed stream and staggered hydrogen and oxygen conduits, where the oxygen feed stream and the hydrogen feed stream flow perpendicular to the propene feed stream.

FIG. 4C is a schematic of a fluidized flow reactor system to produce propylene oxide using the method of the present invention with a propene feed stream and an oxygen feed stream where the oxygen feed stream flows perpendicular to the propene feed stream.

FIG. 5 is a schematic of a continuous flow reactor system to produce propylene oxide using the method of the present invention that includes separated catalytic beds.

FIG. 6A is a schematic of a flow reactor system to produce propylene oxide using the method of the present invention that includes barrier material between each catalytic bed, a propene feed stream, an oxygen feed stream and a hydrogen feed stream.

FIG. 6B is a schematic of a flow reactor system to produce propylene oxide using the method of the present invention that includes barrier material between each catalytic bed, a propene feed stream, an oxygen feed stream.

While the invention is susceptible to various modifications and alternative forms, specific embodiments thereof are shown by way of example in the drawings and may herein be described in detail. The drawings may not be to scale.

### DETAILED DESCRIPTION OF THE INVENTION

The currently available methods to produce propylene oxide from propene are inefficient and suffer from catalyst deactivation. A discovery has been made that allows increased amounts of propene, oxygen, and optionally, hydrogen, to be used in a safe manner in a commercial reactor setting rather than on an experimental reactor setting (e.g., micro reactors). The discovery is premised on the idea of maintaining, in the reaction zone during the reaction, at least 50 vol. % propene, 1 to 15 vol. % O₂, and optionally 1 to 15 vol. % H₂, at high temperatures (e.g., 150° C. to 300° C., preferably 200° C. to 275° C.) and pressures (3 bar to 20 bar, preferably 5 bar to 15 bar) by gradually introducing a feed stream that includes O₂ or multiple feed streams that each include O₂ and H₂ over the length of the catalytic bed or the length of the reaction zone. Without wishing to be bound by theory, it is believed that increasing the concentration of the propene and the oxygen at the catalyst surface will produce more propylene oxide and fewer by-products (e.g., water and/or acrolein). By controlling the position and the addition of the reactant gases to the catalytic bed, the reaction can be run at, outside of, or near the explosive regime for mixtures of propene, oxygen and hydrogen proximate or in the catalytic bed, while not exceeding the explosive concentration at any one point throughout the length of the reaction zone.

These and other non-limiting aspects of the present invention are discussed in further detail in the following sections with reference to the Figures.

### A. Methods for Direct Epoxidation of Propylene

Methods for direct epoxidation of propene are described. The methods can include introducing reactant feeds at different positions and times in the reaction zone such that the total concentration of reactants in the reaction zone are above the explosive regime of the reactant gases, while not exceeding the explosive concentration of the reactant gases at any point throughout the length of the reaction zone. The reaction zone can be a continuous flow reactor selected from a fixed-bed reactor, a fluidized reactor, a plate reactor, a membrane reactor or a moving bed reactor. Non-limiting examples of continuous flow reactors are described in U.S. Pat. No. 6,977,064 to Adris et al., U.S. Pat. No. 7,445,758 to Adris et al., and U.S. Pat. No. 8,288,311 to Dhingra et al. Details about non-limiting reactors are provided in the figures.

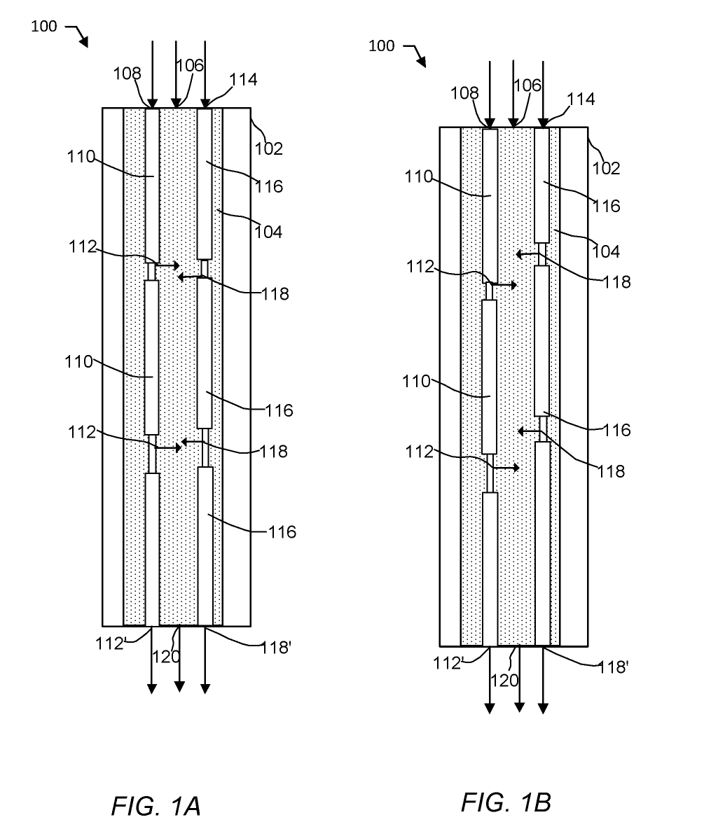

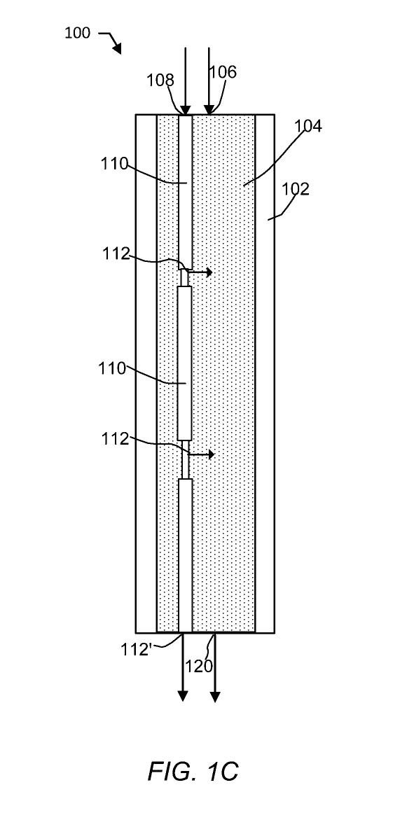

Referring to FIGS. 1A-1C, these figures depict schematics of method 100 in a reaction zone of a reactor for direct epoxidation of propene. FIGS. 1A and 1B illustrate a continuous flow reactor 102 that includes catalyst bed 104, propene feed stream inlet 106, oxygen feed inlet 108, oxygen feed conduit 110, oxygen feed injection outlets 112, 112′, hydrogen feed inlet 114, hydrogen feed conduit 116, hydrogen feed outlets 118, 118′, and product outlet 120. As shown in FIG. 1A, the oxygen and hydrogen feed injection outlets 112 and 118, respectively, are aligned to inject oxygen and/or hydrogen into the same portion of the catalyst bed. As shown in FIG. 1B, the oxygen and hydrogen feed injection outlets are staggered. As shown in FIG. 1C, hydrogen feed conduit is not used. Catalytic bed 104 can include any catalysts suitable for promoting the epoxidation of propylene reaction. In FIGS. 1A-1C, a first feed stream that includes propene can enter catalytic bed via propylene inlet 106. The second feed stream that includes oxygen can enter oxygen conduit 110 via oxygen inlet 108. As shown, the oxygen conduit 110 is positioned in the catalytic bed, however, oxygen conduit 110 can be positioned above or below the catalytic bed. The second feed stream can exit the oxygen conduit via oxygen outlets 112 and 112′. Oxygen outlets 112 provide oxygen to the catalytic bed and can be controlled by one or more flow controllers (e.g., valves or computer controlled valves, not shown). Oxygen not consumed in the reaction can exit the reactor 102 via oxygen outlet 112′. The second stream (oxygen feed stream) can include at any amount of oxygen. In some embodiments at least 20 vol. % to 100 vol. % of oxygen is used. The oxygen outlets 112′ are controlled such that a desired amount of oxygen is fed to a portion of the catalytic bed such that the amount of oxygen is outside the explosive regimen along the length of the catalytic bed (e.g., less than 20 vol. %). For example, 1 vol. % to 15 vol. %, 3 vol. % to 8 vol. %, 4 vol. % to 6 vol. %, or 1 vol. %, 2 vol. %, 3 vol. %, 4 vol. %, 5 vol. %, 6 vol. %, 7 vol. %, 8 vol. % 9 vol. %, 10 vol. %, 11 vol. %, 12 vol. %, 13 vol. %, 14 vol. %, 15 vol. % or any range or value there between of oxygen can be fed to the length of the catalytic bed. In some embodiments, the oxygen can include one or more inert gases. Components of the second feed stream can be obtained from other process units and/or from commercial sources. A third feed stream that includes hydrogen can enter hydrogen conduit 116 via hydrogen inlet 114. Hydrogen conduit 116 can be positioned in, above or below the catalytic bed 104. As shown, the hydrogen conduit 116 is positioned in the catalytic bed. The third feed stream can exit the hydrogen conduit via hydrogen outlets 118 and 118′. Hydrogen outlets 118 provide hydrogen to the catalytic bed and can be controlled by one or more flow controllers (not shown). Hydrogen not consumed in the reaction can exit the reactor 102 via hydrogen outlet 118′. The third stream (hydrogen feed stream) can include any amount of hydrogen. In some embodiments, at least 20 vol. % to 100 vol. % of hydrogen. Hydrogen outlets 118′ are controlled such that a desired amount of hydrogen is fed to a portion of the catalytic bed is outside the explosive regimen along the length of the catalytic bed (e.g., less than 20 vol. %). For example 1 vol. % to 15 vol. %, 3 vol. % to 8 vol. %, 4 vol. % to 6 vol. %, or 1 vol. %, 2 vol. %, 3 vol. %, 4 vol. %, 5 vol. %, 6 vol. %, 7 vol. %, 8 vol. % 9 vol. %, 10 vol. %, 11 vol. %, 12 vol. %, 13 vol. %, 14 vol. %, 15 vol. % or any range or value there between of hydrogen can be fed to the catalytic fed. In some embodiments, the hydrogen stream can include one or more inert gases. Components of the third feed stream (hydrogen feed stream) can be obtained from other process units and/or from commercial sources. In some embodiments, the second and third streams can have a minimal amount of reactant gas or be substantially devoid of reactant gas when exiting the reactor.

In the reaction zone, propene reacts with oxygen to produce propylene oxide. When the catalyst includes gold and titania, hydrogen can react with the gold-titania species to form a peracid species (e.g., Ti—OOH species). The peracid can then react with the olefin to form propylene oxide. In other embodiments, the hydrogen can reduce the metal back to its metal state. In some embodiments, hydrogen is not used (e.g., FIG. 1C). The product stream that includes the propylene oxide can exit the reactor via product outlet 120. The product stream can be collected and/or transported. The product stream can include propene, water and propylene oxide. The product stream can include from at least 1 to 20 vol. %, or 5 to 10 vol. %, or 1 vol. %, 2 vol. %, 3 vol. %, 4 vol. %, 5 vol. %, 6 vol. %, 7 vol. %, 8 vol. %, 9 vol. %, 10 vol. %, 11 vol. %, 12 vol. %, 13 vol. %, 14 vol. %, 15 vol. %, 16 vol. %, 17 vol. %, 18 vol. %, 19 vol. %, 20 vol. % or more, or any value or range there between, of propylene oxide The propylene oxide can be separated from the water and propene using known separation methods.

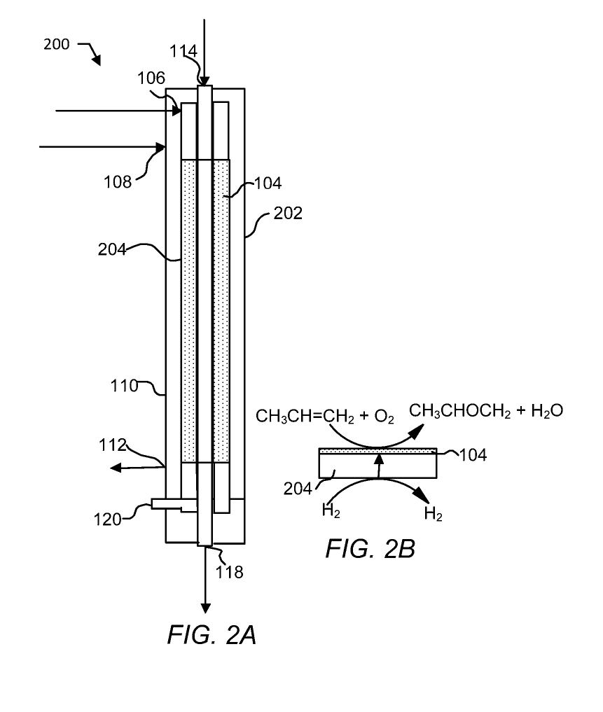

Referring to FIGS. 2A and 2B, method of direct epoxidation of propene is described using a membrane reactor. A non-limiting example of a membrane reactor is described by Oyama et al., Journal of Catalysis, 2008, 257, pp. 1-4, which is incorporated herein by reference. Referring to FIG. 2A, a membrane flow reactor 202 can include catalytic bed 104, first feed stream inlet 106, second feed inlet 108, second feed outlet 112, third feed inlet 114, and third feed outlet 118, product outlet 120, and inner tube 204. FIG. 2B is a cross-sectional view of the membrane and the catalyst with the reactants. In system 200, first feed stream (propene feed stream) and the second feed stream (oxygen feed stream) can enter first feed stream inlet on the shell side of membrane flow reactor 202 via first feed inlet 106 and second feed inlet 108. Hydrogen can enter inner tube 204 of the membrane flow reactor via hydrogen inlet 116. The inner tube (reaction zone) of the membrane flow reactor includes the membrane and the catalytic bed. The third feed stream (hydrogen feed stream) is fed to catalytic bed 204 by permeation through the membrane from the tube side, while first and second feed streams (propene and oxygen) are fed from the opposite side (shell side) of the reactor. In some embodiments, the membrane is a material that provides a barrier between the catalyst/reaction zone and the feed tube. The barrier can provide an even and controllable addition of the gas fed over the length of the reactor. Non-limiting examples of barrier materials that can be used in a membrane reactor are sintered metals or porous ceramics, which can be obtained as tubes of plates. The reactants come in contact and react in the catalyst bed. As shown, the third feed stream (hydrogen) is fed to the reactor through the membrane, however, it should be understood, that both the second and third feed streams could be fed to the catalytic bed through a two membranes respectively.

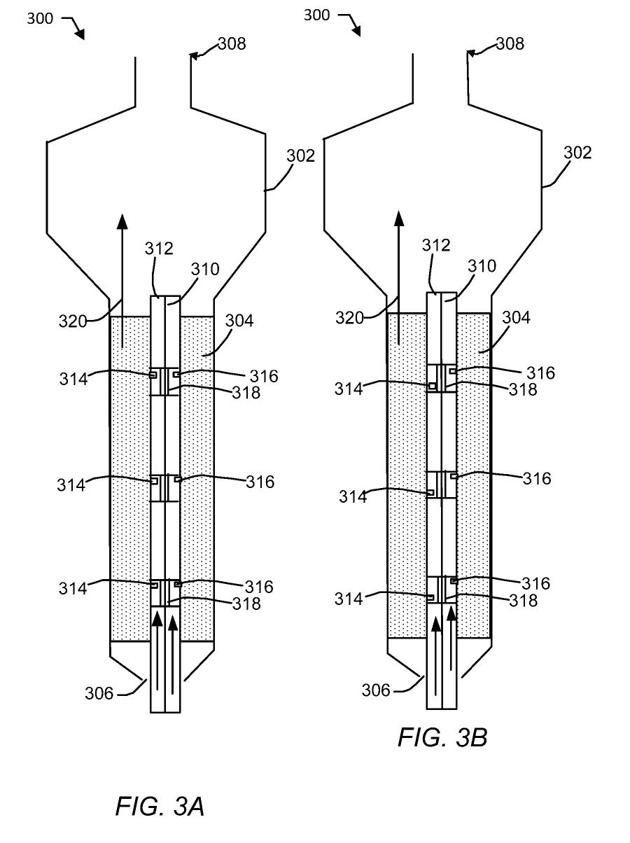

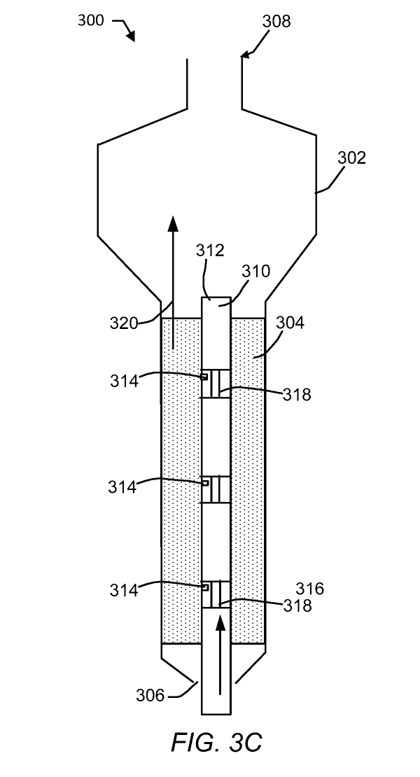

Another embodiment of the invention relates to a continuous flow chemical reaction fluidized bed system 300 as shown in FIGS. 3A through 3B. System 300 includes reactor 302 and a catalyst capable of catalyzing a propylene oxide reaction. Reactor 302 includes fluidized catalytic bed 304 having a height and having first feed inlet 306 at a lower end for a first feed stream (propene feed steam) and product outlet 308 for the product stream (propylene oxide feed stream) at an upper end. In some embodiments, the first feed stream can include propene and oxygen. Fluidized catalytic bed 304 can include interior conduits 310 and 312 extending vertically within the fluidized catalytic bed. Conduits 310 and 312 can have a multiplicity of injectors 314 and 316 spaced apart along the length of the conduit, and each of injectors 314, 316 are capable of introducing a controlled amount of the second and third feed streams into the fluidized catalytic bed 304. Conduits 310 and 312 can include one or more pressure drop devices 318. FIG. 3A depicts injectors 314 and 316 aligned and FIG. 3B depicts injectors 314 and 316 staggered. By way of example, the first feed stream can enter first feed inlet 306 and flow up through fluidized catalytic bed 304 as shown by arrow 320. The velocity of the propene feed stream can be sufficient to fluidized catalytic particles in catalytic bed 304. In some embodiments, other carrier or inert gases can be used to fluidize the catalyst in catalytic bed 304. A controlled amount of the second feed stream (oxygen feed stream) can enter catalytic bed 304 through fluid injector 314 positioned closest to product inlet 306 and a controlled amount of the third feed stream (hydrogen feed stream) can enter the catalytic bed through fluid injector 316 positioned closest to the product inlet. As the first feed stream flows up catalytic bed 304, a controlled amount of the second and/or third feed streams enters the catalytic bed through the next series of injectors. The injection of the second and third feed streams can be done in a simultaneous or sequential manner. In some embodiments, hydrogen is not necessary and the second feed stream (oxygen feed stream) is provided through injectors 314 and 316 or only one conduit is used as shown in FIG. 3C. Contact of the propene with the oxygen in the catalytic bed produces the propylene oxide product stream, which exits reactor 302 through product outlet 308. Although, three injectors are shown, it should be understood that multiple injectors spaced apart can be used.

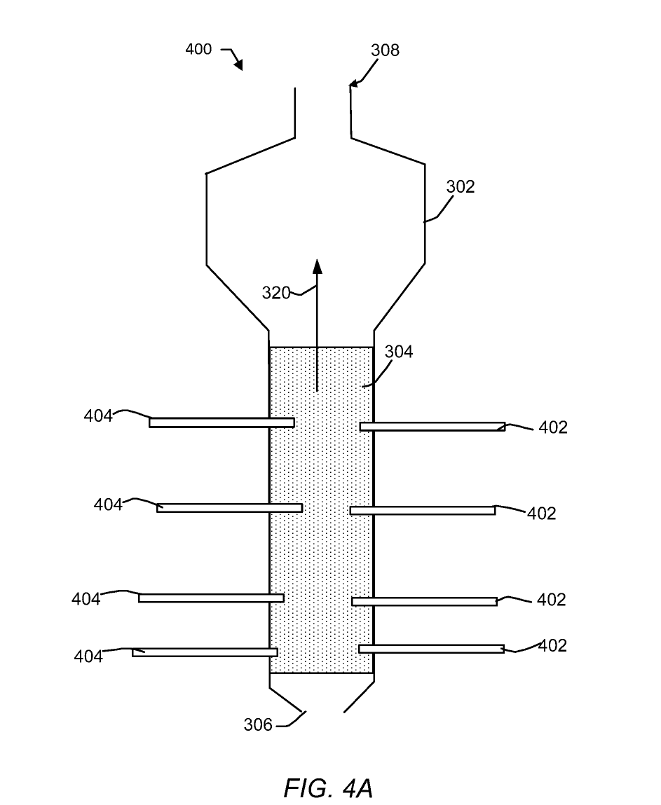

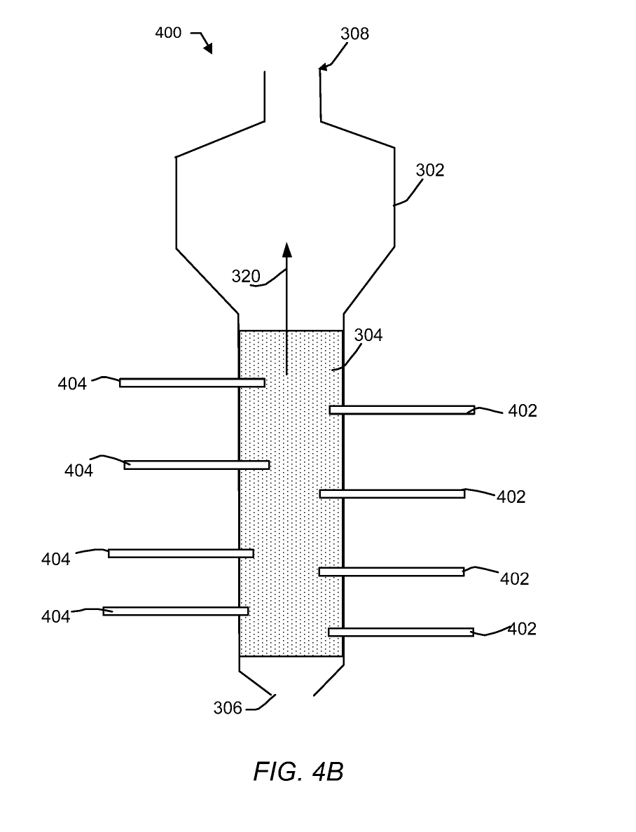

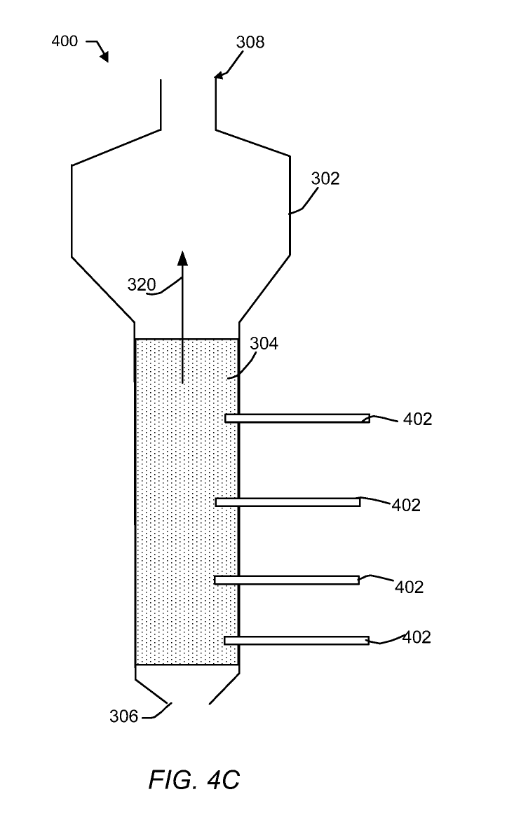

In some embodiments, the conduits that introduce the oxygen and hydrogen into the reactor are perpendicular to the flow of the propylene feed stream. System 400 includes reactor 302 and a catalyst capable of catalyzing a propylene oxide reaction. Reactor 302 includes fluidized catalytic bed 304 having a height and having first feed (propene) inlet 306 at a lower end for a first feed stream (propene feed stream) and product outlet 308 for the product stream (propylene oxide stream) at an upper end. The first feed stream (propene feed stream) can include propene and oxygen. Fluidized catalytic bed 304 can include interior conduits 402 and 404 extending perpendicular within the fluidized catalytic bed. FIG. 4A depicts conduits 402 and 404 aligned. FIG. 4B depicts conduits 402 and 404 staggered. By way of example, the first feed stream can enter first feed inlet 306 and flow up through fluidized catalytic bed 304 as shown by arrow 320. The velocity of the first feed (propene) stream can be sufficient to fluidized catalytic particles in catalytic bed 304. In some embodiments, other carrier or inert gases can be used to fluidize the catalyst in the catalytic bed 304. A controlled amount of the second feed stream (oxygen feed stream) can enter catalytic bed 304 through conduit 402 positioned closest to product inlet 306, and a controlled amount of the third feed stream (hydrogen feed stream) can enter the catalytic bed through conduit 404 positioned closest to product inlet 306. As the first feed stream flows up catalytic bed 304, a controlled amount of the second and/or third feed streams can enter the catalytic bed through the next series of conduits. The injection of second and third feed streams can be done in a simultaneous or sequential manner. In some embodiments, hydrogen is not necessary and the second feed stream (oxygen feed stream) is provided through conduits 402 and 404 or only one second feed stream conduit 404 is used as shown in FIG. 4C. Contact of the propene with the oxygen in the catalytic bed produces the propylene oxide product stream, which exits reactor 302 through product outlet 308. Although, three injectors are shown, it should be understood that multiple injectors spaced apart.

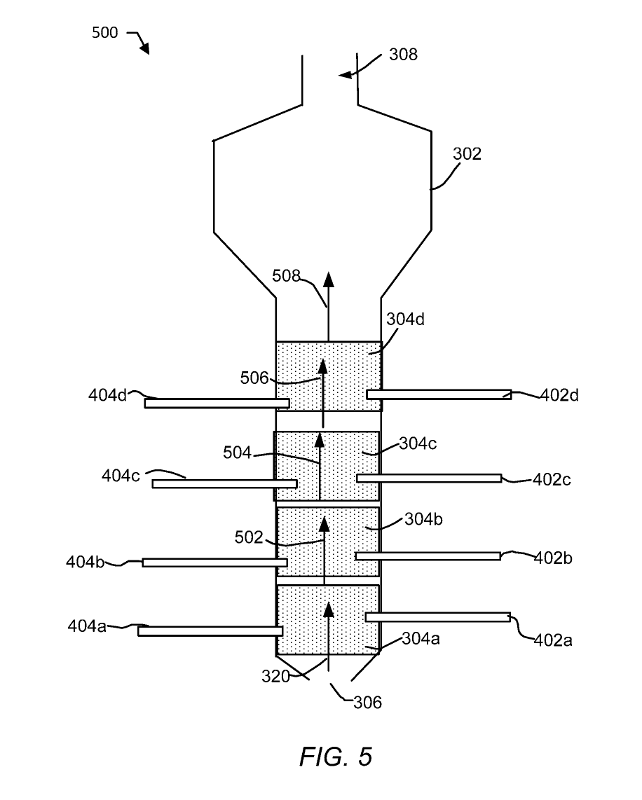

In some embodiments, the catalytic bed in the continuous flow reactor is separated into several catalytic beds. FIG. 5 depicts system 500 that includes fluidized reactor 302 having staggered catalytic beds with conduits 402 and 404 positioned in separated catalytic beds with the second and third streams being provided in a perpendicular flow to the first feed stream. It should be understood that the flow of the second and/or third feed streams can be parallel to the flow of the first feed stream. In FIG. 5, the first feed stream can enter first feed inlet 306 and flow up through fluidized catalytic bed 304a as shown by arrow 320. The velocity of the first feed (propene) stream can be sufficient to fluidized catalytic particles in the catalytic bed. In some embodiments, other carrier or inert gases can be used to fluidize the catalyst in the catalytic bed 304a. A controlled amount of the second feed stream (oxygen feed stream) can enter catalytic bed 304a through conduit 402a, and a controlled amount of the third feed stream (hydrogen feed stream) can enter catalytic bed 304a through conduit 404a. As the first feed stream flows up catalytic bed 304a, the propene reacts with the oxygen to produce first product stream 502. First product stream 502 can include propene, oxygen, hydrogen, propylene oxide and water. First product stream 502 can exit catalytic bed 304a and enter catalytic bed 304b. A controlled amount of the second and/or third feed streams can enter the catalytic bed 304b through conduits 402b and 404b, respectively. The propene in first product stream 502 can react with the oxygen to form second product stream 504 that includes propene, oxygen, hydrogen, propylene oxide and water. Second product stream 504 can exit catalytic bed 304b and enter catalytic bed 304c. A controlled amount of the second and/or third feed streams can enter the catalytic bed 304c through conduits 402c and 404c, respectively. The propene in second product stream 504 can react with the oxygen to form third product stream 506 that includes propene, oxygen, hydrogen, propylene oxide and water. Third product stream 506 can exit catalytic bed 304c and enter catalytic bed 304d. A controlled amount of the second and/or third feed streams can enter the catalytic bed through conduits 402d and 404d, respectively. The propene in third product stream 506 can react with the oxygen to form fourth product stream 508 that includes propene, oxygen, hydrogen, propylene oxide and water. Fourth product stream 508 can exit reactor 302 through outlet 308. The amount of propylene oxide in fourth product stream 508 exiting reactor 302 is greater than the amount of propylene oxide in the first, second and third product streams. The reactor configuration (e.g., interstaged) can allow for tuning of the amount of oxygen and hydrogen to be below the explosive regime of the mixture, while increasing the conversion of the propene to propylene oxide. The injection of second and third feed streams can be done in a simultaneous or sequential manner in any of the catalytic beds. In some embodiments, hydrogen is not injected into one or more of the catalytic beds.

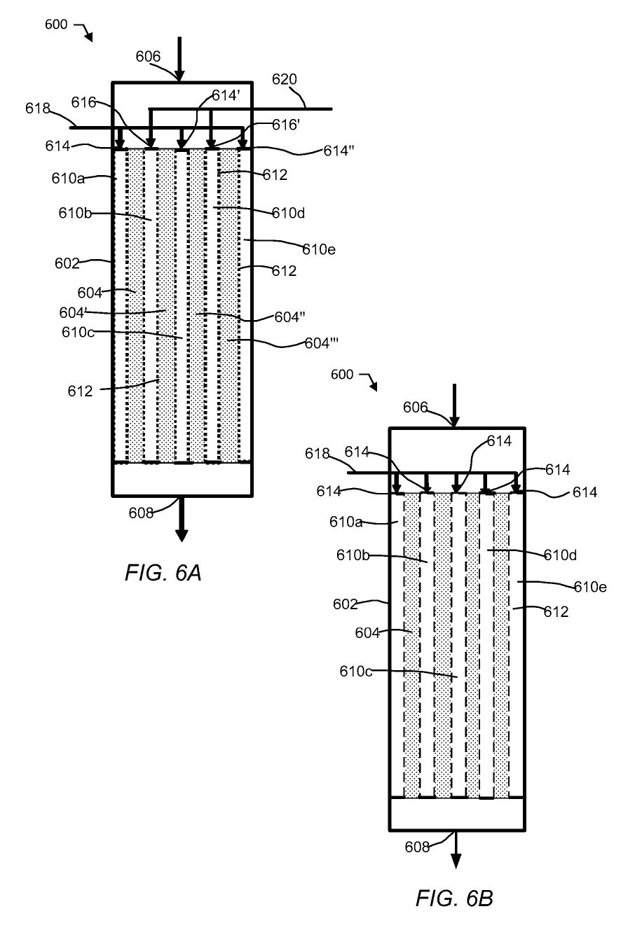

In some embodiments, a porous barrier reactor is used to provide oxygen gas and/or hydrogen gas to the catalytic bed. Porous barrier reactor system 600 is shown in FIGS. 6A and 6B. System 600 can include reactor 602 and catalyst beds 604 (shown as 604, 604′, 604″, and 604′″) that include catalysts capable of catalyzing a propylene oxide reaction. Catalytic beds 604 can have a desired height and/or length and can be isolated from each other by the porous barrier. Each catalytic bed can hold a desired amount of catalyst. In some embodiments, catalytic beds 604 are trays of catalysts. Reactor 602 can include first feed (propene) inlet 606 for a first feed stream (propene feed stream) and product outlet 608 for the product stream (propylene oxide stream) at a reactor end opposite inlet 606. In some embodiments the first feed stream can include propene and oxygen. Reactor 602 can include barrier material 610 (shown as 610a, 610b, 610c, 610d, and 610e). Barrier material 610 can serve as a barrier between the catalyst/reaction zone and the oxygen and/or hydrogen source. Barrier material 610 can be a porous material that allows diffusion of the gas into each of the catalytic beds. As shown, barrier material 610 can include pores 612. Although the pores 612 are shown in FIGS. 6A and 6B, it should be understood that the pores may be microscopic in size. Oxygen and/or hydrogen can enter barrier material (plate or tube) 610 and then diffuse out through pores 612 into catalytic beds 604. The barrier material can provide an even and controllable addition of the gas fed over the length of the reactor. Non-limiting examples of barrier materials that can be used in reactor 602 are sintered metals or porous ceramics, which can be obtained as tubes or plates. As the propene flows over the barrier material, a controlled amount of hydrogen and oxygen are delivered to the catalytic site. Such control of catalyst and reactants inhibits by-product formation and exothermic reactions. A controlled amount of the second feed stream (oxygen feed stream) can enter catalytic beds 604 through oxygen inlets 614, 614′, 614″ or any combination thereof, that are in fluid communication with barrier material 610a, 610c and 610e, respectively. A controlled amount of the third feed stream (hydrogen feed stream) can enter catalytic beds 604 via inlets 616 and/or 616′ that are in fluid communication with barrier material 610b and 610d, respectively. FIG. 6B depicts the reactor system without the hydrogen feed stream. Oxygen can be delivered to barrier material 610a-610e through inlets 614. Manifolds 618 and 620 can include one or more valves to control the flow to the various barrier materials. For example, the second feed stream can be provided to barrier material 610a at a time different than flow provided to other barrier material (e.g., 610c or 610e in FIG. 6A or 610b-610e in FIG. 6B) in reactor 604 and the third feed stream can be simultaneously fed to barrier material 610b (FIG. 6A). In some embodiments, the feed streams are fed simultaneously to all barrier materials and/or at staggered times. The amount of oxygen and/or hydrogen can be determined by monitoring the amount and type of products produced from product outlet 608.

In FIGS. 1-6 reactors 102, 202, 302, 502 and 602 can include one or more heating and/or cooling devices (e.g., insulation, electrical heaters, jacketed heat exchangers in the wall) or controllers (e.g., computers, flow valves, automated values, etc.) that are necessary to control the reaction temperature and pressure of the reaction mixture. While only one reactor is shown, it should be understood that multiple reactors can be housed in one unit or a plurality of reactors housed in one heat transfer unit. Conditions for the propylene epoxidation reaction include a temperature of 150° C. to 300° C., or 150° C., 155° C., 160° C., 165° C., 170, ° C. 175° C., 180° C., 185° C., 190° C., 195° C., 200° C., 205° C., 210° C., 215° C., 220° C., 225° C., 230° C., 235° C., 240° C., 245° C., 250° C., 255° C., 260° C., 265° C., 270° C., 275° C., 280° C., 285° C., 290° C., 295° C., 300° C., or any value or range there between. A pressure of the reaction zone can range from 0.3 MPa to 2 MPa (3 bar to 20 bar), or 0.4 MPa, 0.5 MPa, 0.6 MPa, 0.7 MPa, 0.8 MPa, 0.9 MPa, 1.0 MPa, 1.1 MPa, 1.2 MPa, 1.3 MPa, 1.4 MPa, 1.5 MPa, 1.6 MPa, 1.7 MPa, 1.8 MPa, 1.9 MPa, 2.0 MPa, or any range or value there between. A weight hourly space velocity (WHSV) of propylene can range between 1 and 200 h⁻¹, or 1, 10, 20, 30, 40, 50, 60, 70, 80, 90, 100, 110, 120, 130, 140, 150, 160, 170, 180, 190, 200 h⁻¹ or any range or value there between. WHSV of oxygen and/or hydrogen can be adjusted to make up for the consumed hydrogen and oxygen during the reaction. The WHSV of the hydrogen and oxygen can be less than or approximate the WHSV of the propene entering the reactor.

### B. Materials

Any catalyst capable of catalyzing the propylene oxide reaction can be used. One or more of the catalysts of the current embodiments can include a supported catalyst or bulk metal catalyst that contains metals (e.g., metals in reduced form), metal compounds (e.g., metal oxides) or mixtures thereof (collectively “metals”) of gold (Au), silver (Ag), titanium (Ti), platinum (Pt), palladium (Pd) or combinations thereof. The amount of catalytic metal to be used can depend, inter alia, on the catalytic activity of the catalyst. In some embodiments, the amount of catalytic metal present in the catalyst can range from 0.01 to 100 parts by weight of catalytic metal per 100 parts by weight of catalyst, from 0.01 to 5 parts by weight of catalytic metal per 100 parts by weight of catalyst. If more than one catalytic metal is used, the molar percentage of one metal can be 1 to 99 molar % of the total moles of catalytic metals in the catalyst. The metals can be supported on silica dioxide (SiO₂) or a support that includes Ti in the crystalline SiO₂ structure. Non-limiting examples of such supports include, zeolites TS-1 or Ti-Beta, which can be obtained commercially or manufactured.

The components of the first, second and third feed streams can be obtained from other process units and/or from commercial sources. The propene feed stream (first feed stream) can include at least 50 vol. %, or 82 vol. % to 95 vol. %, or 88 vol. % to 92 vol. % or 50 vol. %, 51 vol. %, 52 vol. %, 53 vol. %, 54 vol. %, 55 vol. %, 56 vol. %, 57 vol. %, 58 vol. %, 59 vol. %, 60 vol. %, 61 vol. %, 62 vol. %, 63 vol. %, 64 vol. %, 65 vol. %, 66 vol. %, 67 vol. %, 68 vol. %, 69 vol. %, 70 vol. %, 71 vol. %, 72 vol. %, 73 vol. %, 74 vol. %, 75 vol. %, 76 vol. %, 77 vol. %, 78 vol. %, 79 vol. %, 80 vol. %, 81 vol. %, 82 vol. %, 83 vol. %, 84 vol. %, 85 vol. %, 86 vol. %, 87 vol. %, 88 vol. %, 89 vol. %, 90 vol. %, 91 vol. %, 92 vol. %, 93 vol. %, 94 vol. %, 95 vol. %, or any value or range there between of propene with the balance being an inert gas, oxygen, hydrogen or combinations thereof. In some instances, gases inert to the propylene oxide reaction can be mixed with the propene. Such gases include carbon dioxide, nitrogen, helium or argon or combinations thereof. The oxygen stream (second feed stream) can include at least 50 vol. %, or 82 vol. % to 95 vol. %, or 88 vol. % to 92 vol. % or 50 vol. %, 51 vol. %, 52 vol. %, 53 vol. %, 54 vol. %, 55 vol. %, 56 vol. %, 57 vol. %, 58 vol. %, 59 vol. %, 60 vol. %, 61 vol. %, 62 vol. %, 63 vol. %, 64 vol. %, 65 vol. %, 66 vol. %, 67 vol. %, 68 vol. %, 69 vol. %, 70 vol. %, 71 vol. %, 72 vol. %, 73 vol. %, 74 vol. %, 75 vol. %, 76 vol. %, 77 vol. %, 78 vol. %, 79 vol. %, 80 vol. %, 81 vol. %, 82 vol. %, 83 vol. %, 84 vol. %, 85 vol. %, 86 vol. %, 87 vol. %, 88 vol. %, 89 vol. %, 90 vol. %, 91 vol. %, 92 vol. %, 93 vol. %, 94 vol. %, 95 vol. %, or any range or value there between of oxygen with the balance being a gas inert to the system (e.g., carbon dioxide, nitrogen, helium, or argon). The third stream (hydrogen feed stream) can include at least 50 vol. %, or 82 vol. % to 95 vol. %, or 88 vol. % to 92 vol. % or 50 vol. %, 51 vol. %, 52 vol. %, 53 vol. %, 54 vol. %, 55 vol. %, 56 vol. %, 57 vol. %, 58 vol. %, 59 vol. %, 60 vol. %, 61 vol. %, 62 vol. %, 63 vol. %, 64 vol. %, 65 vol. %, 66 vol. %, 67 vol. %, 68 vol. %, 69 vol. %, 70 vol. %, 71 vol. %, 72 vol. %, 73 vol. %, 74 vol. %, 75 vol. %, 76 vol. %, 77 vol. %, 78 vol. %, 79 vol. %, 80 vol. %, 81 vol. %, 82 vol. %, 83 vol. %, 84 vol. %, 85 vol. %, 86 vol. %, 87 vol. %, 88 vol. %, 89 vol. %, 90 vol. %, 91 vol. %, 92 vol. %, 93 vol. %, 94 vol. %, 95 vol. %, or any range or value there between of hydrogen with the balance being an inert gas (e.g., carbon dioxide, nitrogen, helium, or argon). In some embodiments, the reactant gas streams include water vapor. In some embodiments, the second and third streams can have a minimal amount of reactant gas or be substantially devoid of reactant gas when exiting the reactor. In some embodiments, the delivery of the hydrogen and/or oxygen is adjusted based on the total amount of propylene, hydrogen and/or oxygen in the catalytic bed or the reactor. The delivery of the hydrogen and/or oxygen can be sequential and/or simultaneous through the various injectors and/or conduits. The flowrate of these streams can be tuned to exactly make up the hydrogen and oxygen consumed by the reaction, so that concentrations of the gases is outside of the explosive regime over the entire reactor length, while keeping these concentrations as high as allowed and not letting hydrogen or oxygen be depleted too much.

The product stream can include hydrogen, propylene oxide, water, alkenes and, in some instances, epoxide ring opening by-products. As described above the hydrogen and alkene oxides can be separated from the product stream. These propylene oxide can be isolated, sold or used in a variety of chemical applications. For example, propylene oxide can be used to make polyether polyols, propylene glycols, and propylene glycol ethers. Propylene oxides can also be used in the manufacture of flame retardants, modified carbohydrates, synthetic lubricants, oil field drilling chemicals, textile surfactants and the like.

### EXAMPLES

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

Computer simulated calculations based on the reaction kinetics of the epoxidation and water formation reactions were performed to compare a conventional method of producing propylene oxide from propylene, hydrogen and oxygen using a single stage reactor without interstaged feed addition and the method of the present invention using a 3-stage reactor with interstaged feed addition. For all the calculations, the kinetic rate expressions used were of a catalyst similar to the one reported by Chen et al. (Chem. Cat. Chem. 2013, 5, 467-478), which was a 0.05 wt % gold catalyst on an amorphous silica support on which 0.2 wt % Ti is deposited.

### Example 1

### Calculations for Producing Propylene Oxide Using the Method of the Present Invention

A 3 stage reactor was modeled. In each reactor stage 5 gram of catalyst was loaded. Operating conditions included: a pressure of 10 bar(a) and a reaction temperature of 483 K (210° C.). The feed rate to the first reactor was 1.12×10⁻⁴ mol/s of propene (152 Nml/min), 6.21×10⁻⁶ mol/s of hydrogen (8.5 N ml/min) and 6.21×10⁻⁶ mol/s of oxygen (8.5 Nml/min). The product composition leaving this reactor was: 1.10×10⁻⁴ mol/s of propene, 2.58×10⁻⁶ mol/s of hydrogen, 3.42×10⁻⁶ mol/s of oxygen, 1.95×10⁻⁶ mol/s of propene oxide, and 3.63×10⁻⁶ mol/s of water. This corresponded to a propene oxide yield (based on propene) of 1.75% and a hydrogen utilization efficiency of 53.9%.

Before the second stage, the hydrogen and oxygen feed streams were supplemented to the original molar flow rates of 6.21×10⁻⁶, i.e. feeding 1.10×10⁻⁴ mol/s propene, 6.21×10⁻⁶ mol/s hydrogen, 6.21×10⁻⁶ mol/sec oxygen, 1.95×10⁻⁶ mol/s propene oxide, and 3.63×10⁻⁶ mol/s of water. The product composition of the 2nd stage was: 1.08×10⁻⁴ mol/s of propene, 2.52×10⁻⁶ mol/s of hydrogen, 3.37×10⁻⁶ mol/s of oxygen, 3.93×10⁻⁶ mol/s of propene oxide, and 7.32×10⁻⁶ mol/s of water. This corresponded to a propene oxide yield (based on propene) of 3.51% and a hydrogen utilization efficiency of 53.6%.

Finally, before the third stage, the hydrogen and oxygen feed streams are again supplemented to the original molar flow rates of 6.21×10⁻⁶ mol/s, i.e. feeding 1.08×10⁻⁴, 6.21×10⁻⁶, 6.21×10⁻⁶, 3.93×10⁻⁶, and 7.32×10⁻⁶ mol/s for propene, hydrogen, oxygen, propene oxide and water. The product composition of the 3rd stage was: 1.06×10⁻⁴ mol/s of propene, 2.45×10⁻⁶ mol/s of hydrogen, 3.33×10⁻⁶ mol/s of oxygen, 5.93×10⁻⁶ mol/s of propene oxide, and 1.11×10⁻⁵ mol/s of water. This corresponded to a final propene oxide yield (based on propene) of 5.30% and a hydrogen utilization efficiency of 53.4%.

### Comparative Example 1

### Calculations for Producing Propylene Oxide Using a Conventional Method

A single reactor containing 15 gram of catalyst was modeled taken without any interstaged feed addition. The original feed of the first reactor described above was used and the hydrogen and oxygen added before the subsequent reactors were omitted to prevent mixing of explosive gas compositions. The feed rate to the reactor was 1.12×10⁻⁴ mol/s (152 Nml/min) of propene, 6.21×10⁻⁶ mol/s of hydrogen (8.5 Nml/min) and 6.21×10⁻⁶ mol/s of oxygen (8.5 Nml/min). The product composition leaving this reactor is: 1.08×10⁻⁴ mol/s of propene, 8.58×10⁻⁸ mol/s of hydrogen, 1.40×10⁻⁶ mol/s of oxygen, 3.49×10⁻⁶ mol/s of propene oxide and 6.13×10⁻⁶ mol/s of water. This corresponded to a propene oxide yield (based on propene) of 3.12% and a hydrogen utilization efficiency of 57.0%.

From the calculations, it can be concluded that the staged reactor method resulted in a significant improvement of the product yield in a same sized reactor with an identical amount of catalyst (5.30% yield versus 3.12% yield). Furthermore, in the comparative single reactor, after 15 grams of catalyst, the hydrogen provided was almost entirely consumed, providing a larger amount of catalyst will therefore not increase the propene oxide yield, whereas in the method of the present invention, a higher conversion can be obtained by adding subsequent staged (i.e. after a 4^{th} reactor containing another 5 grams of catalyst would result in a 7.11% propene oxide yield at 53.3% hydrogen efficiency).

1. A method for direct epoxidation of propene, the method comprising reacting, in a reaction zone of a reactor, propene, oxygen gas (O₂), and hydrogen gas (H₂) in the presence of a catalytic bed that includes a propene epoxidation catalyst to produce a product stream comprising propylene oxide, wherein:
at least 50 vol. % propene, 1 to 15 vol. % O₂, and 1 to 15 vol. % H₂ is maintained in the reaction zone during the reaction by (i) introducing the propene through a first reactant feed stream and (ii) gradually introducing the O₂ or the H₂, or both, over the length of the catalytic bed or the length of the reaction zone through a separate reactant feed stream(s), and
a temperature of 150° C. to 300° C. and a pressure of 3 bar to 20 bar is maintained in the reaction zone during the reaction.

2. The method of claim 1, wherein 82 vol. % to 95 vol. % of propene, 3 vol. % to 8 vol. % O₂, and 2 vol. % to 10 vol. % H₂ is maintained in the reaction zone during the reaction.

3. The method of claim 2, wherein 88 vol. % to 92 vol. % of propene, 4 vol. % to 6 vol. % O₂, and 4 vol. % to 6 vol. % H₂ is maintained in the reaction zone during the reaction.

4. The method of claim 1, wherein the vol. % of propene and O₂ or H₂, or both, in the reaction zone has an explosive regime, and wherein the gradual introduction of O₂ or H₂, or both, in the reaction zone is such that an explosive concentration of propene and O₂ or H₂, or both, is not exceeded at any point throughout the length of the reaction zone.

5. The method of claim 1, wherein a temperature of 200° C. to 275° C. and a pressure of 5 bar to 15 bar is maintained in the reaction zone during the reaction.

6. The method of claim 1, wherein O₂ or H₂, or both, are introduced incrementally over the length of the catalytic bed or the length of the reaction zone.

7. The method of claim 1, wherein O₂ or H₂, or both, are introduced intermittently over the length of the catalytic bed or the length of the reaction zone.

8. The method of claim 1, wherein H₂ is introduced gradually over the length of the catalytic bed or the length of the reaction zone in a second reactant feed stream through a membrane that is positioned proximate to the catalytic bed, and/or O₂ is introduced over the length of the catalytic bed or the length of the reaction zone in a third feed stream through a second membrane that is positioned proximate to the catalytic bed.

9. The method of claim 8, wherein O₂ is introduced with the propene in the first reactant feed stream.

10. The method of claim 1, wherein O₂ is introduced gradually over the length of the catalytic bed or the length of the reaction zone in a second reactant feed stream through a membrane that is positioned proximate to the catalytic bed and/or H₂ is introduced with the propene in the first feed stream.

11. The method of claim 1, wherein the first reactant feed stream is introduced through an inlet positioned upstream from the catalytic bed.

12. The method of claim 1, wherein the separate reactant feed stream that gradually introduces O₂ or H₂, or both, over the length of the catalytic bed or the length of the reaction zone is introduced through a second inlet that is positioned downstream from the first inlet.

13. The method of claim 12, wherein the hydrogen gas is gradually introduced over the length of the catalytic bed or the length of the reaction zone through a second inlet that is positioned downstream from the first inlet and/or the oxygen gas is introduced over the length of the catalytic bed or the length of the reaction zone through a third inlet that is positioned downstream from the first inlet.

14. The method of claim 1, wherein the reactor comprises alternating plates or tubes of an oxygen feed plate or tube, an epoxidation catalyst plate or tube, and a hydrogen feed plate or tube.

15. The method of claim 1, wherein the reaction zone is a continuous flow reactor selected from a fixed-bed reactor, a fluidized reactor, a plate or tube reactor, a membrane reactor or a moving bed reactor.

16. A method for direct epoxidation of propene without using hydrogen gas (H₂) as a reactant, the method comprising reacting, in a reaction zone of a reactor, propene and oxygen gas (O₂) in the presence of a catalytic bed that includes a propene epoxidation catalyst to produce a product stream comprising propylene oxide, wherein:
at least 50 vol. % propene and 1 to 15 vol. % O₂ is maintained in the reaction zone during the reaction by (i) introducing the propene through a first reactant feed stream and (ii) gradually introducing the O₂ over the length of the catalytic bed or the length of the reaction zone through a separate reactant feed stream;
a temperature of 150° C. to 300° C. and a pressure of 3 bar to 20 bar is maintained in the reaction zone during the reaction; and
the direct epoxidation of propene reaction is performed in the absence of H₂ gas.

17. The method of claim 16, wherein the vol. % of propene and O₂ in the reaction zone has an explosive regime, and wherein the gradual introduction of O₂ in the reaction zone is such that an explosive concentration of propene and O₂ is not exceeded at any point throughout the length of the reaction zone.

18. The method of claim 16, wherein O₂ is introduced incrementally or intermittently over the length of the catalytic bed or the length of the reaction zone, or wherein O₂ is introduced gradually over the length of the catalytic bed or the length of the reaction zone in a second reactant feed stream through a membrane that is positioned proximate to the catalytic bed.

19. The method of claim 16, wherein the first reactant feed stream is introduced through an inlet positioned upstream from the catalytic bed and/or the separate reactant feed stream that gradually introduces O₂ over the length of the catalytic bed or the length of the reaction zone is introduced through a second inlet that is positioned downstream from the first inlet.

20. The method of claim 16, wherein the reactor comprises alternating plates or tubes of an oxygen gas feed plate or tube and an epoxidation catalyst plate or tube.
